(19) United States
(12) Patent Application Publication
(10) Pub. No.: US 2017/0340579 A1
(43) Pub. Date: Nov. 30, 2017

(54) TRANSDERMAL DELIVERY SYSTEM

(71) Applicant: SUN PHARMACEUTICAL INDUSTRIES LTD., Mumbai (IN)

(72) Inventors: Madhusudhan BOMMAGANI, Baroda (IN); Pijush Kanti MIDYA, Baroda (IN); Krunal RAVAL, Baroda (IN); Subhas Balaram BHOWMICK, Baroda (IN); Prashant KANE, Baroda (IN)

(73) Assignee: SUN PHARMACEUTICAL INDUSTRIES LTD., Mumbai (IN)

(21) Appl. No.: 15/606,261

(22) Filed: May 26, 2017

(30) Foreign Application Priority Data
May 27, 2016 (IN) 201621018369

(51) Int. Cl. A61K 9/70; B65D 81/26; A61K 31/4458; A61F 15/00; A61J 1/00

(52) U.S. Cl. CPC A61K 9/7053; B65D 81/268; A61K 31/4458; A61J 1/00; A61K 9/70; A61F 15/001; A61K 9/7023

(57) ABSTRACT

A transdermal delivery system comprising a drug-containing matrix layer, a release liner and a backing layer. The drug-containing matrix layer further comprises a methylphenidate base, an adhesive polymer made up of a styrene rubber block copolymer having a styrene content of 24% or above by weight of the adhesive polymer; the adhesive polymer is present in an amount of 20% to 45% by weight of the drug-containing matrix layer, a tackifier present in an amount of 30% to 45% by weight of the drug-containing matrix layer, and a hydrocarbon plasticizer present in an amount of 1% to 30% by weight of the drug-containing matrix layer.

### FIELD OF THE INVENTION

The present invention relates to a transdermal delivery system of methylphenidate.

### BACKGROUND OF THE INVENTION

Methylphenidate transdermal patch is used in Attention Deficit Hyperactivity Disorder (ADHD). This is generally used in children and adolescents having an age group of 6 to 17 years. The commercially marketed transdermal patch of methylphenidate is known by the brand name Daytrana® and is available in various strengths such as 10 mg/9 hr, 15 mg/9 hr, 20 mg/9 hr and 30 mg/9 hr. The composition of the patch has been described in the U.S. Pat. No. 6,210,705. The package insert of Daytrana® provides instructions to the users that the patch should be applied to the hip area two hours before an effect is needed and should be removed nine hours after its application. If a shorter duration of effect is desired or late day side effects appear, it is recommended to remove the patch earlier than nine hours. The transdermal patch presents with the problem of patch locking, i.e. the release liner is not easily removable from the product. This problem increases over time as the peel force required to remove the release liner from the patch increases over time and the patient is unable to remove the release liner as required for use. After application of the patch to the skin, issues such as irritation, reddening and sensitization of skin during the treatment duration have been reported. The U.S. Food and Drug Administration has issued a warning about long term use of the Daytrana™ patch. Long term use of the Daytrana™ patch for periods ranging from 2 months to 4 years has presented with problems of permanent loss of skin color known as chemical leukoderma. This condition is not thought to be reversible and may cause emotional distress to the patients. Furthermore, the patient experiences pain upon removal of the patch. The present inventors have engineered a novel transdermal delivery system of methylphenidate to address the problems discussed above and have emerged with a gentler and improved transdermal delivery system. The present inventors have developed a transdermal delivery system with desirable patch characteristics such as balanced tack, adhesion, cohesion and plasticity.

### SUMMARY OF THE INVENTION

The present invention provides a transdermal delivery system comprising
I. a drug-containing matrix layer comprising:
   a) methylphenidate base,
   b) an adhesive polymer made up of a styrene rubber block copolymer having a styrene content of 24% or above by weight of the adhesive polymer; the adhesive polymer is present in an amount of 20% to 45% by weight of the drug-containing matrix layer,
   c) a tackifier present in an amount of 30% to 45% by weight of the drug-containing matrix layer,
   d) a hydrocarbon plasticizer present in an amount of 1% to 30% by weight of the drug-containing matrix layer,
II. a release liner and
III. a backing layer.

Also the invention provides a transdermal delivery system consisting of
I. a drug-containing matrix layer consisting of:
   a. methylphenidate base,
   b. an adhesive polymer made up of one or more styrene rubber block copolymers having a styrene content of 24% or above by weight of the adhesive polymer; the adhesive polymer is present in an amount of 20% to 45% by weight of the drug-containing matrix layer,
   c. a tackifier present in an amount of 30% to 45% by weight of the drug-containing matrix layer,
   d. a hydrocarbon plasticizer present in an amount of 1% to 30% by weight of the drug-containing matrix layer.
II. a release liner and
III. a backing layer.

### DESCRIPTION OF THE FIGURES

FIG. 1 and FIG. 2 give a representation of the phase separated structure of the rubber midblocks and polystyrene endblocks of the styrene rubber block copolymers.

### DETAILED DESCRIPTION OF THE INVENTION

The transdermal delivery system of the present invention comprises a drug containing matrix layer, a release liner and a backing layer.

The drug-containing matrix layer of the transdermal delivery system comprises methylphenidate base. In the preferred embodiments, it is present in the range of 10% to 40% more preferably in the range of 15% to 30%, such as for example 16%, 17%, 18%, 19%, 20%, 21%, 22%, 23%, 24%, 25%, 26%, 27%, 28%, 29% or 30% by weight of the drug-containing matrix layer. In one preferred embodiment, methylphenidate is present in the transdermal delivery system in an amount of 20% by weight of the drug-containing matrix layer.

The drug-containing matrix layer of the transdermal delivery system comprises one or more styrene rubber block copolymers as an adhesive polymer. The styrene rubber copolymers include but are not limited to rubber copolymers of styrene-butadiene (SB), styrene-isoprene (SI), styrene-butadiene-styrene (S-B-S), styrene-isoprene-styrene (SIS), styrene-(ethylene/butylene)-styrene (SEBS) or styrene-(ethylene/propylene)-styrene (SEPS) and mixtures thereof. Compared to typical plastics or commercial rubbers (homopolymers or random copolymers), they have a unique molecular structure. They are composed of pure blocks of each monomer with an elastomeric rubber block in the center called as the midblock polymer and a hard polystyrene block on the end of each polymer chain called the endblock polymer. The polymers can be chemically represented as:

TABLE 1
Chemical composition of styrene rubber block copolymers

| | Chemical |
|---|---|
| Structures | composition |
| 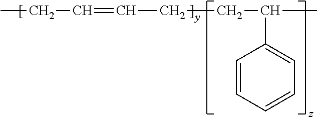 | Styrene butadiene copolymer |
| 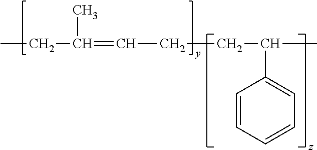 | Styrene isoprene copolymer |
| 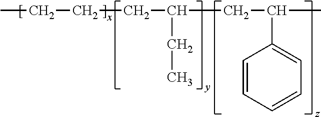 | Ethylene/ Butylene/ Styrene Copolymer |
| 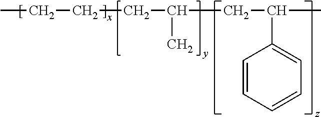 | Ethylene/ Propylene/ Styrene Copolymer |

Polymers product line includes diblock, triblock, radial and star polymer structures and each structure contributes unique properties to the polymer. The different types of styrene rubber copolymers can be structurally presented as below:

TABLE 2
Different types of styrene rubber copolymers

| Polymer Type | Structure | Chemistry | Presentation |
|---|---|---|---|
| SBS | Linear | —[C—C═C—C]ₙ— | S—B • B—S |
| SBS | Diblock/Linear | Polybutadiene [B] | S—B |
| (SB)ₙ | Radial | | 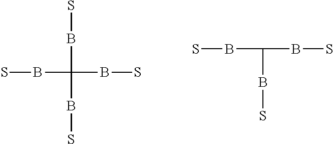 |
| SIS SIS | Linear Diblock/Linear | 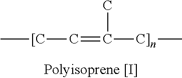 | S—I • I—S S—I |
| (SI)ₙ | Radial | | 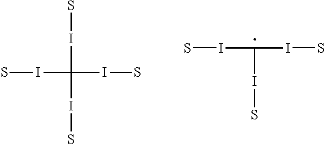 |
| (SI)ₙ | Star | | 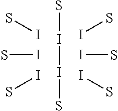 |
| SEBS SEBS | Linear Diblock/Linear | 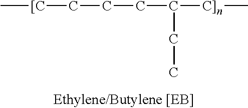 | S—EB • EB—S S—EB |
| SEP | Diblock | 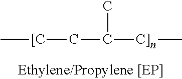 | S—EP |
| (SEP)ₙ | Star | | 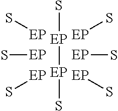 |

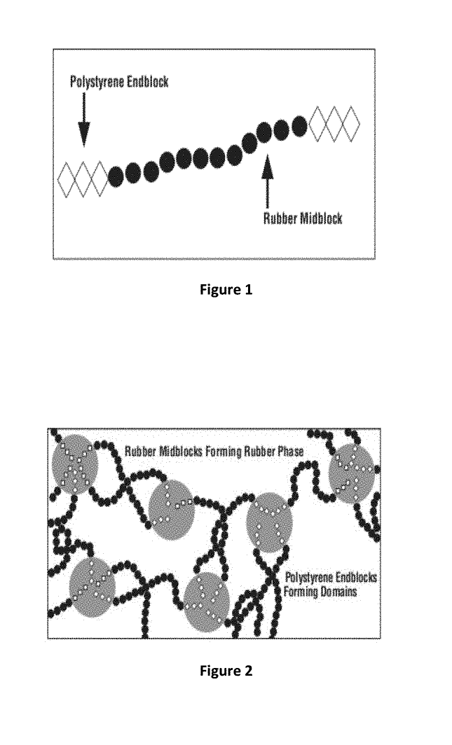

Each type of polymer has two glass transition temperatures (Tg) for two blocks—an upper one of about 95° C. for the polystyrene end block and a lower one for the rubber midblock of −85° C. for the B (butadiene) block; −60° C. for the I (isoprene) block, and −55° C. for EB (ethylene/butylene) and EP (ethylene/propylene) blocks and both blocks remain thermodynamically incompatible. The two-phase structure gives their high strength at end-use temperatures and also provides low viscosity and easy processing at elevated temperatures or in solvent solutions. A representation of this phase separated structure of the rubber midblocks and polystyrene end blocks is presented in the FIGS. 1 and 2.

In one embodiment, the adhesive polymer used in the drug containing matrix layer of the present invention is styrene butadiene copolymer or styrene butadiene styrene copolymer having a styrene content of 24% or above by weight of the styrene rubber block copolymer, preferably 25% or above, more preferably 28% or above, preferably in the range of 25 to 55%, more preferably in the range of 30 to 50% and most preferably a styrene content in the range of 32 to 42% by weight of the styrene rubber block copolymer. The commercially available styrene butadiene styrene copolymers which can be used include but are not limited to DURO-TAK 6911, Kraton D 1118, Kraton D0243, Kraton D 1155, Kraton D1133, Kraton D1102 and the like.

In one embodiment, the adhesive polymer used in the drug containing matrix layer of the present invention is styrene isoprene copolymers or styrene-isoprene-styrene copolymers having a styrene content of 24% or above by weight of the styrene rubber block copolymer, preferably 25% or above, more preferably 28% or above, preferably in the range of 25 to 50%, more preferably in the range of 25 to 45% and most preferably in the range of 27 to 44% by weight of the styrene rubber block copolymer. The commercially available styrene-isoprene-styrene copolymers which can be used include but are not limited to Kraton D1162, Kraton D1165, Kraton D 1164, Kraton™ D1102, Kraton D1193 and the like.

In another embodiment, the adhesive polymer used in the drug containing matrix layer of the present invention is styrene ethylene/butylene-styrene copolymers having a styrene content of 24% or above by weight of the styrene rubber block copolymer, preferably 25% or above, more preferably 27% or above, preferably in the range of 25 to 65%, more preferably 28 to 60% and most preferably in the range of 30 to 58% by weight of the styrene rubber block copolymer. The commercially available styrene ethylene/butylene-styrene copolymers which can be used include but are not limited to Kraton A1535H, Kraton A1536 H, Kraton G1633, Kraton G1650, Kraton G1651, Kraton G1652, Kraton G165, Kraton G1660, SEPTON™ 8004, SEPTON™ 8006, SEPTON™ 8007, SEPTON™ 8076 and the like.

In another embodiment, the adhesive polymer used in the drug containing matrix layer of the present invention is styrene-ethylene/propylene-styrene copolymers having a styrene content of 24% or above by weight of the styrene rubber block copolymer, preferably 25% or above, more preferably 28% or above, preferably in the range of 25 to 40%, more preferably in the range of 26 to 38% and most preferably in the range of 28 to 37% by weight of the styrene rubber block copolymer. The commercially available styrene-ethylene/propylene-styrene copolymers which can be used include but are not limited to Kraton G1701 and Kraton G1702, SEPTON™ 2002, SEPTON™ 2006, SEPTON™ 2104, SEPTON™ 4033, SEPTON™ 4044, SEPTON™ 4055, SEPTON™ 4077, SEPTON™ 4099 and the like. In some embodiments a mixture of one or more of the styrene rubber block copolymers as described herein may be used. Preferably, in one or more embodiments, the drug containing matrix layer is free of acrylate based adhesive polymers.

The amount of the styrene rubber block copolymer used in the present invention as the adhesive polymer is in the range of 10-70%, preferably 15-60%, more preferably 18-50%, more preferably in the range of 20% to 45% by weight, such as for example 21, 22, 23, 24, 25, 26, 27, 28, 29, 30, 31, 32, 33, 34, 35, 36, 37, 38, 39, 40, 41, 42, 43, 44 or 45% by weight, most preferably 25% to 35% by weight of the drug-containing matrix layer. In particularly preferred embodiments, the SIS copolymer Kraton D1162 having a styrene content of 44% by weight is used as an adhesive polymer and it is preferably used in an amount of 25% to 35% by weight of the drug-containing matrix layer.

The drug-containing matrix layer of the transdermal delivery system of the present invention contains one or more tackifiers. The tackifiers which may be used within the scope of the present invention include, but are not limited to, aliphatic hydrocarbons, mixed aliphatic and aromatic hydrocarbons, aromatic hydrocarbons, substituted aromatic hydrocarbons, hydrogenated esters, polyterpenes, and hydrogenated wood rosins. In some embodiments, the polymer matrix includes one or more tackifiers selected from rosin esters, aliphatic hydrocarbon resins, aromatic hydrocarbon resins, terpene resins, polybutene, and hydrogenated polybutene and a number of other commercially available tackifiers or mixtures thereof. In specific embodiments, the polymer matrix includes one or more C₅ to C₉ Alicyclic saturated hydrocarbon resins (HHR) (Arkon P 100). The tackifier is present in an amount in the range of 30 to 45% by weight, preferably 30.0 to 40.0% by weight of the drug-containing matrix layer, such as for example 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5 or 40.0% by weight of the drug containing matrix layer. In some preferred embodiments, the tackifier is present in an amount in the range of 31.0 to 39.0% by weight, preferably 32.0 to 38.0% by weight of the drug containing matrix layer. It was found that when the tackifier concentration was about 48% (i.e. outside the claimed range of the invention), and all other components being in the desired claimed range, the patch showed cohesive failure and while removing the patch, a very high tack force was needed for debonding as well as a mass was left on the skin.

The drug containing matrix layer of the transdermal delivery system comprises one or more plasticizers. The preferable plasticizer for use with styrene rubber block copolymer is one that is completely insoluble in the endblock polystyrene phase and completely miscible with the midblock rubber phase of the block copolymer. The plasticizer which is used in the present invention includes a hydrocarbon and are referred to herein as hydrocarbon plasticizers. The hydrocarbon plasticizer which can be used within the scope of this invention include but are not limited to mineral oil, light mineral oil, isoparaffin, petroleum derivative wax, paraffin, soft paraffin, white soft paraffin, yellow soft paraffin, polyisobutylene and the like or mixtures thereof. The plasticizer is used in the drug containing matrix layer in an amount in the range of 1 to 30% by weight of the drug-containing matrix layer, preferably in the range of 2 to 25%, such as for example 3, 4, 5, 6, 7, 8, 9, 10, 11, 12, 13, 14, 15, 16 ,17, 18, 19, 20, 21, 22, 23, 24 or 25%, more preferably in the range of 10 to 25% and most preferably in the range of 12 to 20% by weight of the drug-containing matrix layer. In one embodiment, the plasticizer is used in the range of 2 to 8% by weight of the drug-containing matrix layer. In another preferred embodiment the plasticizer is used in the range of 15 to 25% by weight of the drug containing matrix layer. It was found that when a patch was prepared without use of a plasticizer (while all other components being in desired claimed range), the patch lead to painful peel off from the skin.

The drug containing matrix layer of the transdermal delivery system may optionally contain one or more permeation enhancers, fillers and other additives or excipients known for use in the transdermal delivery system. The permeation enhancers which may be included within the scope of the present invention include but are not limited to lauryl alcohol, citric acid triethyl ester, myristic acid isopropyl ester, lactic acid cetyl ester, oleyl alcohol, sorbitan monoolate, polyethylene glycol monostearate, lauromacrogol, N-methyl-2-pyrrolidone, triacetin, pyrrothiodecane, sodium acetate, etc. The permeation enhancers may be used in the range of 1 to 10% by weight of the drug-containing matrix layer.

The transdermal delivery system of the invention is prepared by forming the drug-containing matrix layer on a release liner and then forming a backing layer thereon. For the release liner, conventional release liners or their laminates used in the field of transdermal delivery system may be used. For example, a film, a paper, or laminates thereof, which is made of polyethylene, polyester, polyvinyl chloride, polyvinylidene chloride, etc. coated with silicone resin or fluoride resin may be used. In one preferred embodiment the release liner is silicone-coated polyester liner commercially known as 75 mic PET Clear C1S-E as a one side direct silicone coated, transparent PET liner with easy release value of 5-25 g/inch which works as a protective release liner. Also, drug non-absorbable and flexible materials conventionally used in the field of transdermal delivery system may be used as the backing layer (also referred to as “backing membrane”). For example, polyolefin, polyethylene, polyether, a multi-layer ethylene vinyl acetate film, polyester, polyurethane, and the like may be used. In one preferred embodiment, the polyester/ethylene vinyl acetate laminate is used as the backing layer commercially known as 3M Scotchpak 9733™.

The transdermal delivery system of the present invention is in the form of a patch and the performance characteristics of the patch can be determined by various tests which check the adequacy of adhesive, cohesive properties of the patch. The peel adhesion properties are tested by measuring the force required to pull an adhesive coated tape, applied to the substrate at 90° angle and additionally, deposition of adhesive residue on the substrate is noted down. Tack properties include the ability of the polymer to adhere to substrate with little contact pressure. It is measured by different approaches such as thumb tack test, rolling ball method, quick stick method, and probe tack methods. The force required to remove thumb from adhesive is measured in Thumb tack method. Rolling ball method involves measurement of the distance that a stainless steel ball travels along an upward facing adhesive. The less tacky the adhesive, the further the ball will travel. In quick stick (Loop tack) method, the pull force required to break the bond between an adhesive and substrate is measured by pulling the tape away from the substrate at 90° at the speed of 12 inch/min. The probe tack test measures the force required to pull a probe away from an adhesive at a fixed rate. (Fred H. Hammond, Jr. 1989, Chapter 4: Tack, Handbook of pressure sensitive adhesive technology, 2nd edition, Rhode Island: Satas & Associates. pp. 38-39 and Wokowich et al, Transdermal drug delivery system (TDDS) adhesion as a critical safety, efficacy and quality attribute, European Journal of Pharmaceutics and Biopharmaceutics 64 (2006) 1-8)). The adhesion and cohesion characteristics of the transdermal system of the present invention may be determined by various techniques known in the art. Cohesive failure is defined as some part of the adhesive material of the transdermal delivery system remaining on the skin after peeling off of the transdermal system. The addition of tackifier in the aforementioned range is important to give a patch that does not show any adhesion failure during the wearing period of patch i.e. the patch adheres well to the skin. Adhesion failure is defined as a matrix that does not have sufficient adhesion, which may cause the patch to be removed prematurely during the treatment duration. Adhesive material of transdermal system for skin application must be fairly soft with a low modulus of elasticity. The modulus of elasticity is defined as the tendency of an object to deform along an axis when opposing forces are applied along that axis. It is the ratio of tensile stress to tensile strain. Adhesive material with a high elastic modulus debonds easily upon continuous flexing of skin during wearing period of patch and also is more liable to cause mechanical skin irritation. The addition of plasticizer in the range mentioned before gives a patch favorable for comfortable wearing as well as smoothly peeling the patch off from the skin.

In one instance the adhesion character of the transdermal delivery system is determined by the peel test that determines the resistance to peel by measuring the force required to peel away a strip of tape from a rigid surface. Peel resistance data yields more information about the adhesive character and its expected performance than other commonly used tests of pressure sensitive adhesives used in transdermal delivery systems. In one or more embodiments, the in-vivo tack property of the formulation is studied by thumb tack method.

The transdermal delivery system of methylphenidate according to the present invention when tested for various performance characteristics according to one or more methods described above, was found to possess optimum, balanced peel adhesion properties, tack properties, adhesion and cohesion characteristics and plasticity. Particularly, in specific embodiment, the methylphenidate transdermal system having a combination of styrene rubber copolymer as the adhesive polymer with a styrene content of 24% or above by weight of the polymer present in an amount of 20% to 45% by weight of the drug-containing matrix layer, a tackifier in the range of 30 to 45% by weight of the drug-containing matrix layer and a hydrocarbon plasticizer, preferably mineral oil in the range of 1 to 30% by weight of the drug-containing matrix layer, a release liner and a backing layer, produced a patch with the most balanced tack, cohesion, adhesion properties and plasticity. The styrene rubber block copolymer, tackifier and mineral oil used in the aforementioned ranges in various permutations and combinations provides a patch with satisfactory cohesion and adhesion properties. The transdermal delivery system (patch) according to the present invention show no problem of patch locking and the release liner gets easily removed from the product while use. Also the patch can be conveniently applied and removed from the skin without any pain upon removal of the patch. This happens because the transdermal delivery system of the present invention possesses optimum, desirable patch characteristics such as balanced tack, adhesion, cohesion and plasticity.

The styrene rubber block copolymers used as adhesive polymers according to the present invention essentially have styrene content of 24% or above, preferably 25% or above, preferably in the range of 25-65% by weight of the styrene rubber block copolymer. Such polymers impart desired adhesive and cohesive characteristics to the methylphenidate patch. It was found that when styrene rubber block copolymers having lower styrene content (lower than 24%) were used as adhesive polymers, the resulting patch did not show desired cohesive and adhesive properties. Particularly, the patch fails in cohesive test, i.e. it did not have sufficient cohesion and some adhesive mass remain on the skin after removal of the patch.

In one embodiment, the present invention provides a transdermal delivery system comprising a drug-containing matrix layer, a release liner and a backing layer wherein the drug containing matrix layer comprises methylphenidate base present in an amount of 10% to 40% by weight of the drug-containing matrix layer; an adhesive polymer made up of a styrene rubber block copolymer having a styrene content of 25% or above by weight of the adhesive polymer, the adhesive polymer is present in an amount of 20% to 45% by weight of the drug-containing matrix layer; a tackifier present in an amount of 30% to 45% by weight of the drug-containing matrix layer; and a hydrocarbon plasticizer present in an amount of 1% to 30% by weight of the drug-containing matrix layer.

In one embodiment, the present invention provides a transdermal delivery system comprising a drug-containing matrix layer, a release liner and a backing layer, wherein the drug containing matrix layer consist of methylphenidate base, an adhesive polymer made up of a styrene rubber block copolymer having a styrene content of 24% or above by weight of the adhesive polymer present in an amount of 20% to 45% by weight of the drug-containing matrix layer, a tackifier present in an amount of 30% to 45% by weight of the drug-containing matrix layer and a hydrocarbon plasticizer present in an amount of 1% to 30% by weight of the drug-containing matrix layer.

In preferred embodiment, the present invention provides a transdermal delivery system comprising a drug-containing matrix layer, a release liner and a backing layer wherein the drug containing matrix layer comprises methylphenidate base present in an amount of 10% to 30% by weight of the drug-containing matrix layer; an adhesive polymer made up of a styrene rubber block copolymer having a styrene content of 25-65% by weight of the adhesive polymer, the adhesive polymer is present in an amount of 20% to 39% by weight of the drug-containing matrix layer; a tackifier present in an amount of 31% to 39% by weight of the drug-containing matrix layer; and a hydrocarbon plasticizer present in an amount of 2% to 25% by weight of the drug-containing matrix layer.

In preferred embodiment, the present invention provides a transdermal delivery system comprising a drug-containing matrix layer, a release liner and a backing layer wherein the drug containing matrix layer comprises methylphenidate base present in an amount of 15% to 25% by weight of the drug-containing matrix layer; an adhesive polymer made up of a styrene rubber block copolymer having a styrene content of 25-65% by weight of the adhesive polymer, the adhesive polymer is present in an amount of 25% to 35% by weight of the drug-containing matrix layer; a tackifier present in an amount of 32% to 38% by weight of the drug-containing matrix layer; and a hydrocarbon plasticizer present in an amount of 10% to 25% by weight of the drug-containing matrix layer.

The transdermal delivery system, in the form of a patch is packed into a primary pouch formed from primary packaging film and sealed to form a pouch referred to herein as the primary packaging. The primary packaging film comprises but are not limited to polyethylenes, polyesters, polyethylene terephthalate (PET), polypropylenes, polyurethanes, polyolefin, polyvinyl alcohol, polyvinyl chloride, polyvinylidene, polyamide, vinyl acetate resins, ethylene/vinyl acetate copolymers, ethylene/ethylacrylate copolymers, polyacrylonitriles (PAN), polyethylene terephthalate aluminum foil, high molecular weight polyethylene terephthalate (HMPET), ethylene vinyl alcohol copolymers metal-vapor deposited films or sheets thereof, rubber sheets or films, expanded synthetic resin sheets or films, non-woven fabrics, aluminum (Al) foils, papers or combinations thereof. The primary packaging film is generally multilayered. The primary packaging film may be PAN (Barex)/PET laminate pouch which is a polyacrylonitrile layer followed by polyethylene terephthalate layer and further coupled with aluminum lamination. Alternatively, it can be composed of a layer of polyethylene terephthalate (PET) followed by a layer of adhesive further followed by a layer of aluminum (Al) and layer of high molecular weight polyethylene terephthalate (HMPET), suitably the primary packaging film has the specifications of PET 23 μm thickness/Adhesive 2.5 μm thickness/Al 9 μm thickness/Adhesive 2.5 μm thickness/HMPET 50 μm thickness laminate pouch. It may be composed as a layer of polyethylene terephthalate followed by a layer of polyethylene (PE) followed by a layer of oriented polyamide (OPA) followed by a layer of coating of polyethylene terephthalate (LAS/PolE) suitably present as PET 23 μm thickness/PE 10 g/m²/OPA 25 μm thickness/LAS Pol E 16 g/m² laminate pouch. Also it may be present as composed of a polyethylene terephthalate layer followed by a layer of polyethylene followed by a layer of oriented polyamide followed by a layer of polyamide coating (LAS Pol D) suitably present as PET 23 μm thickness/PE 10 g/m²/OPA 25 μm thickness/LAS Pol D 12 gsm) laminate pouch. The primary packaging film can be composed of a layer of polyethylene terephthalate followed by a layer of coating of ethylene vinyl alcohol copolymer coating (LAS Pol B), suitably present as PET 36 μm thickness/LAS Pol B 12 g/m²) laminate pouch. The primary packaging film may be composed of a layer of polyethylene terephthalate and a further coating of ethylene vinyl alcohol copolymer (LAS Pol B) coating suitably present as PET 50 μm thickness/LAS Pol B 12 g/m² laminate pouch to name a few. In one embodiment the transdermal delivery system may be packed in a primary packaging alone comprising a layer of paper followed by a layer of adhesive, followed by a layer of polyethylene terephthalate, again an adhesive laminated with aluminum foil and finally as layer of resistant inert sealant (RIS), presented suitably as Paper 40 gsm/Adhesive 2.5μ/PET 23μ/Adhesive 2.5μ/Al 9μ/RIS 15 (5 gsm) laminate pouch or packed in primary packaging composed of polyethylene terephthalate with a layer of adhesive followed by an aluminum lamination, an adhesive layer again and a final layer of polyacrylonitrile, suitably presented as PET 12μ/adhesive 2.5μ/Aluminum 9μ/Adhesive 2.5μ/PAN 50) laminate pouch or a layer of polyethylene terephthalate followed by a layer of adhesive followed by an aluminum lamination further having an adhesive layer and a final layer of high molecular weight polyethylene terephthalate presented as PET 23μ/Adhesive 2.5μ/Al 9 μ/Adhesive 2.5μ/HMPET 50μ laminate pouch.

The multiple units of primary packaging may be further placed into secondary packaging such as for example secondary packaging pouch. The secondary packaging helps for further protecting the transdermal delivery system from the ingress of moisture and is not directly in contact with the transdermal delivery system. The secondary packaging may additionally comprise a dessicant, which may be present as a pouch or in a dessicant holder or as an inbuild dessicant layer in the secondary packaging pouch. The secondary packaging material is a multilayered film having an outer layer of polyethylene terephthalate (PET) followed by adhesive layer (ADH), followed by an aluminum layer (Al) and finally an inner layer made by co-extruding polyethylene (PE) with desiccant. Suitably the film is PET 12μ/Adh/Al (12μ)/PE 50 gm/m² coextruded with desiccant. The dessicant used in the secondary packaging comprises but is not limited to silica, calcium oxide and the like. Preferably the dessicant used in the secondary packaging is calcium oxide. This inbuilt dessicant layer scavenges residual moisture in the packaging as well as moisture that penetrates through the seal area, known as cross diffusion. The drying capacity guaranteed is 0.25 mg/cm². The secondary packaging material acts as a barrier against light, oxygen and water vapour. It is puncture resistant and has a high resistance to easy opening of the system. The water vapour transmission rate of this secondary packaging material is 0.1 g/m² at 38° C. at 90% HR and the oxygen permeability at 23° C. and 50% HR is less than 0.1 cc/m².day.bar.

In one embodiment, the transdermal delivery system in the form of a patch may be packed into a primary packaging alone. In another embodiment multiple units of the transdermal delivery system packed in the primary packaging are further packed into secondary packaging. In one specific embodiment five units of the transdermal delivery system packed in a primary packaging are further packed into a secondary packaging. This is beneficial to keep the transdermal delivery system stable for a longer duration and can be used upto the expiry date as per patient convenience as against the marketed product Daytrana™. The marketed product is available in a pack of 30 transdermal patches having an inner packing and then put in a single outer pack, due to which once the outer pack is opened, the patches are susceptible to degradation and 30 patches have to be used within the stipulated 2 months duration which can be inconvenient to the patient. In one preferred embodiment the transdermal delivery system of the present invention is packed in a primary packaging of PET 23 μm/PE 10 gsm/OPA 25 μm/LAS Pol D 12 gsm followed by a secondary packaging pouch of laminated polyethylene terephthalate of 12 μm size, an adhesive layer, an aluminum layer of 12 μm size and 50 g/m² weight and polyethylene coextrusion coating with desiccant. The use of a specific combination of primary and secondary packaging helps protect the drug from degradation by prevention of ingress of moisture and oxygen through the packaging.

The transdermal system of the present invention provides a stable patch which gives a reduced impurity profile in comparison to a commercially marketed transdermal patch of methylphenidate base known as Daytrana™. The methylphenidate patch of the present invention on being subjected to stability tests at room temperature for at least 12 months and at accelerated condition of 40° C./75% relative humidity for a period of 3 and 6 months showed lower levels of impurities which are within the desired limit such as for example, erythro acid not more than 0.24% by weight, threo acid not more than 3.5% by weight, erythro ester not more than 2.0% by weight, Impurity C, D, and F not more than 0.24% by weight, highest unknown impurity not more than 0.2% by weight and total impurities not more than 6.0% by weight of methylphenindate. Also, the drug assay level remains within the limit of 90-110% of label claim.

In yet another aspect, the present invention provides a patch with uniform controlled release profile.

It can be concluded that the transdermal delivery system with an adhesive polymer made up of a styrene rubber block copolymer having higher percentage of styrene of 24% or above, methylphenidate as the drug of choice, a tackifier and mineral oil together in the drug containing matrix layer provided an excellent patch with optimal cohesive and adhesive properties. The tackifier resin is useful for providing adequate adhesive properties to the transdermal system. The use of mineral oil decreases the hardness and modulus at room temperature, eliminates drawing, decreases cohesive strength or increases plasticity of the styrene block copolymer and helps the transdermal delivery system to be easily peeled off the skin. The particular combination of the styrene rubber copolymer and methylphenidate, tackifier and mineral oil helps in developing a stable patch for methylphenidate. Furthermore, the use of the unique packaging comprising the primary and secondary packaging helps to make a long term stable transdermal system for methylphenidate.

In the context of this specification “comprising” is to be interpreted as “including”. Aspects of the invention comprising certain elements are also intended to extend to alternative embodiments “consisting” or “consisting essentially” of the relevant elements.

Where technically appropriate, embodiments of the invention may be combined.

Embodiments are described herein as comprising certain features/elements. The disclosure also extends to separate embodiments consisting or consisting essentially of said features/elements.

Any embodiments specifically and explicitly recited herein may form the basis of a disclaimer either alone or in combination with one or more further embodiments.

Hereinafter, the invention will be more specifically described by way of Examples. The examples are not intended to limit the scope of the invention and are merely used as illustrations.

### EXAMPLES 1-13

The drug containing matrix layer of the transdermal delivery system according to the present invention was prepared as per the Examples 1 to 13 described in Table 3 below.

TABLE 3
Composition details of the transdermal delivery system

| Components | | EXAMPLES | | | | | | | | | | | | |
|---|---|---|---|---|---|---|---|---|---|---|---|---|---|---|
| of matrix | | 1 | 2 | 3 | 4 | 5 | 6 | 7 | 8 | 9 | 10 | 11 | 12 | 13 |
| composition | Ingredients | % w/w of drug containing matrix layer on a dry basis | | | | | | | | | | | | |
| Drug | Methylphenidate base | 20 | | | | | | | | | | | | |
| Adhesive | Styrene/Isoprene/styrene | — | — | 28 | 28 | 28 | 28 | 28 | 28 | 28 | 28 | — | — | — |
| polymer | Block Copolymer | | | | | | | | | | | | | |
| | (Kraton D1162) | | | | | | | | | | | | | |
| | Styrene isoprene styrene | — | 28 | — | — | — | — | — | — | — | — | — | — | — |
| | Block copolymer | | | | | | | | | | | | | |
| | (Kraton D1164) | | | | | | | | | | | | | |
| | Styrene isoprene styrene | 28 | — | — | — | — | — | — | — | — | — | — | — | — |
| | Block copolymer | | | | | | | | | | | | | |
| | (Kraton D1165) | | | | | | | | | | | | | |
| | Styrene ethylene | — | — | — | — | — | — | — | — | — | — | 13 | 20 | — |
| | butylene styrene block | | | | | | | | | | | | | |
| | copolymer (Kraton | | | | | | | | | | | | | |
| | A1535 H) | | | | | | | | | | | | | |
| | Styrene butadiene block | — | — | — | — | — | — | — | — | — | — | 25.74 | — | — |
| | copolymer (Polymer of | | | | | | | | | | | | | |
| | DURO-TAK 87-6911) | | | | | | | | | | | | | |
| | Styrene isoprene styrene | — | — | — | — | — | — | — | — | — | — | — | — | 39 |
| | Block copolymer | | | | | | | | | | | | | |
| | (Kraton D1193) | | | | | | | | | | | | | |
| Tackifier | Alicyclic saturated | 35 | 35 | 32.5 | 34 | 33 | 35 | 36 | 37 | 33.5 | 34.5 | — | 36 | 33 |
| | hydrocarbon resin | | | | | | | | | | | | | |
| | (Arkon P100) | | | | | | | | | | | | | |
| | Synthetic hydrocarbon | — | — | — | — | — | — | — | — | — | — | 38.68 | — | — |
| | resin (Tackifier of | | | | | | | | | | | | | |
| | DURO-TAK 87-6911) | | | | | | | | | | | | | |
| Plasticizer | Mineral oil | 17 | 17 | 19.5 | 18 | 19 | 17 | 16 | 15 | 18.5 | 17.5 | 2 | 24 | 8 |

Method of Preparation:
I. Preparation of drug containing matrix layer composition: Toluene, n-heptane and mineral oil were taken in an appropriate container and temperature was maintained at 25±5° C. The alicyclic saturated hydrocarbon resin (Arkon P-100) and styrene rubber block copolymer (Kraton™ or Durotak™) were added slowly under stirring to the solvent and mineral oil mixture and stirred to get clear solution. The specified quantity of methylphenidate base was added slowly under stirring to this solution and stirred to get a clear solution.
II. Coating of drug containing matrix layer onto the release liner and drying and lamination with a backing layer: The drug containing matrix layer was coated onto the release liner on one side and the backing layer on the other end.
III. Die cutting of Transdermal adhesive system: The laminate was cut into a patch with the dimensions of 35 mm×35.7 mm, 49.4 mm×38 mm, 65.8 mm×38 mm and 98.7 mm×38 mm for the 10, 15, 20 & 30 mg/9 hour strengths respectively by using Fly press die cutting machine to form the final patch.
IV. Pouching and Sealing: Each patch was packed in a primary packaging of PAN (Barex)/PET laminate pouch and all the open sides of the pouch sealed using heat sealing machine. Five PAN (Barex)/PET pouches containing the patch pack, were packed in a secondary pack containing inbuilt desiccant laminated pouch and all open sides of the pouch were sealed.

### COMPARATIVE EXAMPLES 1-3

The matrix layers which are not according to the present invention are tabulated below in Table 4. These are mentioned as comparative examples.

TABLE 4
Composition details of comparative examples
of the transdermal delivery system

| Components | | Comparative EXAMPLES | | |
|---|---|---|---|---|
| of matrix | | 1 | 2 | 3 |
| composition | Ingredients | % w/w on a dry basis | | |
| Drug | Methylphenidate base | 20 | | |
| Tackifier | Alicyclic saturated | 32.5 | | |
| | hydrocarbon resin | | | |
| | (Arkon P100) | | | |
| Plasticizer | Mineral oil | 19.5 | | |
| Adhesive | Styrene isoprene styrene | 28 | — | — |
| polymer | block copolymer | | | |
| | (Kraton D 1111) | | | |
| | Styrene isoprene styrene | — | 28 | — |
| | block copolymer | | | |
| | (Kraton D1193) | | | |
| | Styrene isoprene styrene | — | — | 28 |
| | block copolymer | | | |
| | (Kraton D1114) | | | |

The drug containing matrix layer composition of the comparative examples were coated onto the release liner and laminated with the backing layer on the other side and were evaluated for patch performance. They were found to fail in that the patches did not have sufficient cohesion, so some adhesive mass was left on the skin after removal of patch.

### EXAMPLE 14a-14b

TABLE 5
Composition details of the transdermal delivery system

| Components | | | |
|---|---|---|---|
| of matrix | | % w/w on a dry basis | |
| composition | Ingredients | Example 14 a | Example 14 b |
| Drug | Methylphenidate base | 20 | |
| Adhesive | Styrene/Isoprene/styrene | 28 | |
| polymer | Block Copolymer | | |
| | (Kraton D1162) | | |
| Tackifier | Alicyclic saturated | 32.5 | |
| | hydrocarbon resin | | |
| | (Arkon P100) | | |
| Plasticizer | Light mineral oil | 19.5 | — |
| | White soft paraffin | — | 19.5 |

The examples 14a and 14b were prepared as per the process of examples 1 to 13, albeit with the use of different hydrocarbon plasticizers.

### EXAMPLE 15

Peel Adhesion Test: The transdermal delivery systems prepared as per example 1-13 and comparative examples 1-3 and examples 14a and 14b were subjected to the peel adhesion test using the Universal Testing Machine having a stainless steel test panel to determine the force required to peel away the strip. The procedure for this test was as follows:
1) The stainless steel panel was rinsed with acetone and dried with lint free absorbent tissue and allowed to air dry. This procedure was repeated twice.
2) The stainless steel test panel was allowed to dry for at least 10 minutes.
3) Two pieces of Scotch Pak 232 of width 25 mm and length 160 mm were cut from the tape roll to serve the purpose of Tape Leader.
4) The transdermal delivery system (test sample) prepared as per the examples listed before was cut into a width 25 mm and length 50 mm.
5) 5 mm of the release liner of the test sample from one end (across the width) and the tape leader was positioned between the backing film and the release liner such that the tape leader adhesive was facing the backing film. The tape leader was placed on approximately 5 mm of the exposed test sample. The remainder of the tape leader was folded back onto itself (adhesive-to-adhesive) to form a double thickness leader.
6) The release liner was removed from the test sample using teflon coated tweezers and the exposed adhesive of the test sample was placed onto the clean stainless steel test panel.
7) Stainless steel test panel was placed on the 90° peel fixture of Universal Testing machine.
8) Folded end of the tape leader was clamped to the upper grip of crosshead (moving jaw) at 90° peel angle
9) The upward motion of crosshead was started at velocity of 5 mm/s (30 cm/min).
10) After the movable jaw was started in motion, the force values obtained while the first 10 mm of peeled were disregarded and average force obtained during peeling of the next 25 mm was used as the adhesion force value.
11) Adhesive residue observed on SS panel immediately after peeling of tape was recorded.

All examples 1 to 13 and 14a and 14b showed good formulation characteristics such as no mass left on the stainless steel panel, which is an indicator of no cohesive failure at the time of patch removal from skin and no immature adhesive failure during the wearing period of the patch. The comparative examples 1 to 3 had mass left on the stainless steel panel and showed cohesive failure.

### EXAMPLE 16a-16f

The transdermal delivery system units prepared as per example 10 were packaged and sealed in different types of packaging materials. Stability testing was done by storage of the transdermal delivery system at room temperature for 12 months and at accelerated condition of 40° C./75% relative humidity (RH) for 3 month to 6 months. The pack styles and packaging for examples (a) to (f) is as mentioned below:
a) Primary (PET 23 μm/Adhesive 2.5 μm/PAN 25 μm) laminate pouch, those five pouches further packed in a secondary packaging with inbuilt desiccant laminated pouch.
b) Primary (PET 12 μm/adhesive 2.5 μm/Aluminum 9 μm/Adhesive 2.5 μm/PAN 50 μm) laminate pouch.
c) Primary (PET 23 μm/Adhesive 2.5 μm/Al 9 μm/Adhesive 2.5 μm/HMPET 50 μm) laminate pouch.
d) Primary (PET 23 μm/PE 10 gsm/OPA 25 μm/LAS Pol E 16 gsm) laminate pouch, those five pouches further packed in a secondary packaging with inbuilt desiccant laminated pouch.
e) Primary (PET 23 μm/PE 10 gsm/OPA 25 μm/LAS Pol D 12 gsm) laminate pouch, those five pouches further packed in a secondary packaging with inbuilt desiccant laminated pouch.
f) Primary (PET 36 μm/LAS Pol B 12 gsm) laminate pouch, those five pouches further packed in a secondary packaging with inbuilt desiccant laminated pouch.

The results of stability study upon storage at accelerated condition of 40° C./75% relative humidity (RH) are given below in table 6.

TABLE 6
Degradation profile of methylphenidate transdermal patch prepared as per the present
invention and packaged in different packaging materials.

| | | | Degradation Profile (n = 2) | | | | | | | |
|---|---|---|---|---|---|---|---|---|---|---|
| | | | | Impurity A | Impurity B | | | | Highest | |
| | | Assay of | Erythro | (Threo | (Erythro | | | | Unknown | Total |
| | Time | Methylphenidate | Acid | acid) | ester) | Impurity C | Impurity D | Impurity F | Impurity | Impurities |
| | Point | (±RSD) | (Limit: | (Limit: | (Limit: | (Limit: | (Limit: | (Limit: | (Limit: | (Limit: |
| Storage | (M = | (Limit: 90-110%) | NMT | NMT | NMT | NMT | NMT | NMT | NMT | NMT |
| Condition | Month) | (n = 5) | 0.24%) | 3.5%) | 2.0%) | 0.24%) | 0.24%) | 0.24%) | 0.2%) | 6.0%) |

Example 16 a

| | | | | | | | | | | |
|---|---|---|---|---|---|---|---|---|---|---|
| Initial | 0 M | 95.73 (±4.9) | ND | ND | ND | ND | ND | ND | ND | ND |
| 40° C./75% RH | 3 M | 106.2 (±5.2) | ND | ND | 0.102 | ND | ND | ND | ND | 0.102 |

Example 16 b

| | | | | | | | | | | |
|---|---|---|---|---|---|---|---|---|---|---|
| Initial | 0 M | 95.73 (±4.9) | ND | ND | ND | ND | ND | ND | ND | ND |
| 40° C./75% RH | 3M | 93.3 (±4.4) | ND | 0.468 | 0.473 | ND | ND | ND | 0.01 | 0.951 |

Example 16 c

| | | | | | | | | | | |
|---|---|---|---|---|---|---|---|---|---|---|
| Initial | 0 M | 101.72 (±1.5) | ND | ND | ND | ND | ND | ND | 0.008 | 0.008 |
| 40° C./75% RH | 3 M | 97.1 (±2.6) | ND | 1.34 | 0.845 | ND | ND | ND | 0.068 | 2.253 |

Example 16 d to f

| | | | | | | | | | | |
|---|---|---|---|---|---|---|---|---|---|---|
| Initial | 0 M | 104.6 (±0.9) | ND | ND | ND | ND | ND | ND | ND | ND |

Example 16 d

| | | | | | | | | | | |
|---|---|---|---|---|---|---|---|---|---|---|
| 40° C./75% RH | 3 M | 96.7 (±2.2) | ND | 0.054 | 0.19 | ND | ND | ND | 0.042 | 0.30 |

Example 16 e

| | | | | | | | | | | |
|---|---|---|---|---|---|---|---|---|---|---|
| 40° C./75% RH | 3 M | 100.6 (±1.9) | ND | 0.045 | 0.167 | ND | ND | ND | 0.052 | 0.291 |

Example 16 f

| | | | | | | | | | | |
|---|---|---|---|---|---|---|---|---|---|---|
| 40° C./75% RH | 3 M | 104.2 (±1.4) | ND | 0.048 | 0.18 | ND | ND | ND | 0.047 | 0.303 |

NMT = Not more than;
ND = Not Detected

It was found that the compositions of example 16a to 16f when stored at room temperature or at accelerated stability condition i.e. 40° C./75% Relative Humidity for a period of 3 months, the levels of Impurities A to F, highest unknown impurity, impurity Erythro A and total impurities were within the specified limits as provided in Table 6 above. The results indicate that the transdermal delivery system packed in primary packaging alone i.e. examples 16b and 16c showed some loss in drug assay and comparatively higher degradation profile as compared to examples 16a, 16d, 16e and 16f which were packed in a primary packaging plus secondary package with inbuilt desiccant. However, the levels of impurities were within the desired specified limits.

The methylphenidate marketed transdermal system Daytrana® pack of 30 was used as a reference. The outer pack containing 30 patches, each of the 30 patches further packed in an inner packing were tested for stability. Stability was tested at three time points:
I. Patch tested at twelve months before expiry when patch was just opened which was the initial time point,
II. Patch tested at 2 months before expiry when patch was just opened,
III. After having kept the outer packing open for two months and exposing the inner packing containing the patches at 25° C./60% RH.

The results obtained are described in Table 7:

TABLE 7
Degradation profile of the reference methylphenidate transdermal system Daytrana ®

| | Assay of | Degradation Profile (n = 2) | | | | | | | |
|---|---|---|---|---|---|---|---|---|---|
| | Methyl | | | Impurity B | | | | Highest | |
| | phenidate | Erythro | Impurity A | (Erythro | | | | Unknown | Total |
| Condition/ | (±RSD) | Acid | (Threo acid) | ester) | Impurity C | Impurity D | Impurity F | Impurity | Impurities |
| Time | (90-110%); | (NMT | (NMT | (NMT | (NMT | (NMT | (NMT | (NMT | (NMT |
| Point | (n = 5) | 0.24%) | 3.5%) | 2.0%) | 0.24%) | 0.24%) | 0.24%) | 0.2%) | 6.0%) |
| I | 97.71 | ND | 0.659 | 0.096 | ND | ND | ND | ND | 0.755 |
| II | 99.46 | ND | 1.977 | 0.286 | ND | ND | ND | ND | 2.263 |
| III | 98.95 | ND | 2.97 | 0.421 | ND | ND | ND | ND | 3.391 |

The test was done to simulate frequency and duration of use of the Daytrana® patch by patient after opening of the secondary packaging and to check the stability of the patches for the 2 month duration of use by the patient. It was seen from the results that the threo acid impurity, erythro-ester impurity and total impurities of the marketed transdermal system Daytrana® when patch package was kept open for 2 months were higher than the initial time point where the patch package was just opened.

We claim:

1. A transdermal delivery system comprising:
I. a drug-containing matrix layer comprising:
a. methylphenidate base,
b. an adhesive polymer made up of one or more styrene rubber block copolymers having a styrene content of 24% or above by weight of the adhesive polymer; the adhesive polymer is present in an amount of 20% to 45% by weight of the drug-containing matrix layer,
c. a tackifier present in an amount of 30% to 45% by weight of the drug-containing matrix layer,
d. a hydrocarbon plasticizer present in an amount of 1% to 30% by weight of the drug-containing matrix layer,
II. a release liner and
III. a backing layer.

2. The transdermal delivery system as claimed in claim 1, wherein the methylphenidate base is present in the range of 15% to 30% by weight of the drug containing matrix layer.

3. The transdermal delivery system of claim 1, wherein the styrene rubber block copolymer is made up of a group consisting of styrene and isoprene or butadiene or ethylene butylene or ethylene propylene or mixtures thereof.

4. The transdermal delivery system of claim 3, wherein the styrene rubber block copolymer is styrene/isoprene/styrene block copolymer, styrene isoprene block copolymer or mixtures thereof.

5. The transdermal delivery system of claim 3, wherein the styrene rubber block copolymer is styrene butadiene block copolymer, styrene butadiene styrene block copolymer or mixtures thereof.

6. The transdermal delivery system of claim 3, wherein the styrene rubber block copolymer is styrene ethylene butylene styrene block copolymer.

7. The transdermal delivery system of claim 3, wherein the styrene rubber block copolymer is styrene ethylene propylene styrene block copolymer.

8. The transdermal delivery system of claim 1, wherein the tackifier is present in an amount of 30% to 40% by weight of the drug-containing matrix layer.

9. The transdermal delivery system of claim 1, wherein the tackifier is C₅-C₉ alicyclic saturated hydrocarbon resin.

10. The transdermal delivery system of claim 1, wherein the hydrocarbon plasticizer is selected from the group consisting of mineral oil, light mineral oil and white soft paraffin.

11. The transdermal delivery system of claim 1, wherein the transdermal system is packed into a primary packaging and optionally the transdermal system packed in the primary packaging is further packed into a secondary packaging.

12. The transdermal delivery system of claim 1, wherein the transdermal system is
a) packed into a primary packaging.
b) multiple units of primary packaging are further packed into a secondary packaging comprising an inbuilt desiccant.

13. The transdermal delivery system of claim 12, wherein the primary packaging is a laminate pouch comprising polyethylene terephthalate layer followed by a layer of polyethylene followed by a layer of oriented polyamide followed by a layer of polyamide coating.

14. The transdermal delivery system of claim 12, wherein the secondary packaging consists of polyethylene terephthalate, an adhesive layer, an aluminum layer and a polyethylene co-extrusion coating with a desiccant.

15. The transdermal delivery system of claim 12, wherein five units of the primary packaging are further packed into a secondary packaging comprising an inbuilt desiccant.

16. A transdermal delivery system consisting of:
I. a drug-containing matrix layer consisting of:
a. methylphenidate base,
b. an adhesive polymer made up of one or more styrene rubber block copolymers having a styrene content of 24% or above by weight of the adhesive polymer; the adhesive polymer is present in an amount of 20% to 45% by weight of the drug-containing matrix layer,
c. a tackifier present in an amount of 30% to 45% by weight of the drug-containing matrix layer,
d. a hydrocarbon plasticizer present in an amount of 1% to 30% by weight of the drug-containing matrix layer.
II. a release liner and
III. a backing layer.

17. The transdermal delivery system as claimed in claim 16, wherein the transdermal system is
a) packed into a primary packaging.
b) multiple units of primary packaging are further packed into a secondary packaging comprising an inbuilt desiccant.
